(19) United States
(12) Patent Application Publication
(10) Pub. No.: US 2025/0071878 A1
(43) Pub. Date: Feb. 27, 2025

(54) LIGHTING APPARATUS

(71) Applicant: LEEDARSON LIGHTING CO.,LTD., Zhangzhou (CN)

(72) Inventors: Pingqiu Lin, Zhangzhou (CN); Dianqiu Zhang, Zhangzhou (CN); Zebin Ye, Zhangzhou (CN); Weijun Lin, Zhangzhou (CN)

(21) Appl. No.: 18/811,619

(22) Filed: Aug. 21, 2024

(30) Foreign Application Priority Data
Aug. 22, 2023 (CN) 202322262386.0

(51) Int. Cl. H05B 47/19; A61L 9/20; F04D 25/08; F21V 14/02; F21V 29/71; F21V 33/00; F21Y 115/10; H05B 45/37; H05B 47/17

(52) U.S. Cl. CPC H05B 47/19; A61L 9/20; F04D 25/088; F21V 14/02; F21V 29/713; F21V 33/0096; H05B 45/37; H05B 47/17; F21Y 2115/10

(57) ABSTRACT

A lighting apparatus includes a fan, a light device, an integrated housing, a main controller, a fan controller, a light controller and a converter. The integrated housing is used for disposing the fan and the light device. The converter converts an external AC power to a direct current. The fan controller is coupled to the converter for generating a fan driving current to the fan according to a fan control signal from the main controller. The light controller is coupled to the converter for generating a light driving current to the light device according to a light control signal. The main controller receives a wireless main control signal from a wall switch operated by a user. The main controller generates the fan control signal and the light control signal based on the wireless main control signal.

### FIELD

The present invention is related to a lighting apparatus, and more particularly related to a lighting apparatus with an integrated fan.

### BACKGROUND

The development of LED (Light Emitting Diode) light devices has revolutionized the lighting industry, becoming the preferred choice for a wide range of applications. The journey of LED technology from niche uses to mainstream lighting solutions is a testament to the innovation and adaptability of these devices. LEDs are highly energy-efficient, consuming significantly less power than traditional incandescent bulbs while offering a longer lifespan. This efficiency has made them popular in both residential and commercial settings, contributing to substantial energy savings and a reduction in carbon footprints.

One of the key reasons for the widespread adoption of LED lights is their versatility. Unlike traditional lighting, which often comes in limited shapes and sizes, LEDs can be engineered in various forms, from small indicator lights to large panels. This flexibility allows for a broad spectrum of applications, including decorative lighting, task lighting, and even horticultural lighting. The ability to design LEDs in different colors without the need for filters also adds to their popularity, enabling dynamic and mood-enhancing lighting solutions.

Despite their advantages, the evolution of LED lighting continues as consumer and industrial needs grow more sophisticated. For instance, there is a growing demand for LED lights with added functionality, such as smart control features that allow users to adjust brightness, color temperature, and even lighting schedules through smartphones or voice commands. This integration with smart home systems is particularly appealing to tech-savvy consumers who seek convenience and customization in their lighting solutions.

Another area where LED lighting is evolving is in ease of installation. Consumers are increasingly looking for plug-and-play solutions that do not require professional installation. This demand has led to the development of more user-friendly products, such as adhesive-backed LED strips, modular lighting systems, and wireless options that can be easily set up and controlled without complex wiring. This trend towards simplicity is crucial in making advanced lighting technologies accessible to a broader audience.

The need for flexible settings in LED lighting is also growing. In commercial and residential environments, lighting requirements can vary significantly depending on the time of day, the activity being performed, or the mood the space should evoke. Adjustable LED lights that can change color temperature from warm to cool light, or that can be dimmed to various levels, are increasingly in demand. These features not only enhance the user experience but also contribute to energy efficiency by providing the right amount of light when and where it is needed.

Balancing the diverse needs of consumers and businesses while pushing the boundaries of LED technology is challenging. Manufacturers must consider factors such as cost, energy efficiency, ease of use, and the aesthetic appeal of their products. For instance, integrating advanced features like Wi-Fi control or motion sensors can drive up the cost, potentially limiting accessibility for some consumers. However, the continuous innovation in this space suggests that manufacturers are finding ways to offer these features without compromising affordability.

The widespread use of lighting in daily life means that even small improvements in LED technology can have a significant impact. For example, the development of LEDs that emit less blue light can contribute to better sleep patterns for users, addressing concerns about the impact of light exposure on circadian rhythms. Similarly, innovations that enhance the longevity and durability of LEDs can reduce waste and lower the environmental impact of lighting.

Furthermore, the integration of LEDs with renewable energy sources is another exciting development. Solar-powered LED lights, for instance, are becoming increasingly common, especially in outdoor applications. These lights provide a sustainable option for reducing energy consumption while offering the same, if not better, performance as traditional lighting systems. The marriage of LED technology with renewable energy sources underscores the potential for these lights to play a pivotal role in achieving global sustainability goals.

As LED technology continues to evolve, we are likely to see even more innovative applications that go beyond basic lighting. Concepts like human-centric lighting, which adjusts based on the time of day to support human health and well-being, are gaining traction. Similarly, LEDs integrated with sensors and AI could lead to more intelligent lighting systems that adapt to the user's needs without manual intervention, providing optimal lighting conditions in real-time. Each innovation in this field not only enhances the functionality of lighting but also contributes to the broader goal of improving the quality of life through better, smarter lighting solutions.

Ceiling fans have long been a staple in home and commercial spaces, offering an efficient way to circulate air and maintain a comfortable environment. These fans are particularly popular because they can help reduce the need for air conditioning by promoting better airflow. By moving air throughout a room, ceiling fans create a wind-chill effect, making the space feel cooler even if the actual temperature remains unchanged. This effect allows occupants to set their thermostats a few degrees higher without sacrificing comfort, which can lead to significant energy savings.

In addition to their cooling capabilities, ceiling fans are also effective in the winter months. Many modern ceiling fans come with a reversible motor feature that allows the blades to spin in the opposite direction. When set to rotate clockwise at a low speed, ceiling fans can help distribute warm air that naturally rises to the ceiling, pushing it back down into the living space. This can help maintain a more consistent temperature throughout the room and reduce heating costs.

Ceiling fans are also valued for their aesthetic versatility. They are available in a wide range of styles, sizes, and finishes, making it easy to find a fan that complements the décor of any room. Whether in a minimalist, modern space or a more traditional setting, ceiling fans can serve both a functional and decorative purpose. Many fans also come equipped with integrated lighting, allowing them to serve a dual role as both a fan and a light fixture, further enhancing their utility.

The popularity of ceiling fans is also due to their ability to improve air quality. By continuously moving air, ceiling fans can help prevent the buildup of stagnant air and reduce the accumulation of dust and allergens. This is particularly beneficial in rooms where ventilation might be limited, as the constant air movement helps to ensure a fresh and healthy environment. For individuals with allergies or respiratory issues, the regular use of ceiling fans can contribute to a more comfortable living space.

Finally, the ease of installation and the long-term durability of ceiling fans contribute to their enduring popularity. Unlike other cooling systems that may require complex installation and maintenance, ceiling fans are relatively straightforward to install and maintain. They are designed to last for many years, making them a cost-effective investment for homeowners and businesses alike. With the ability to enhance comfort, reduce energy costs, and add aesthetic value, ceiling fans continue to be a popular choice for climate control in a wide range of settings.

### SUMMARY

In some embodiments, a lighting apparatus includes a fan, a light device, an integrated housing, a main controller, a fan controller, a light controller and a converter.

The integrated housing is used for disposing the fan and the light device.

The converter converts an external AC power to a direct current.

The fan controller is coupled to the converter for generating a fan driving current to the fan according to a fan control signal from the main controller.

The light controller is coupled to the converter for generating a light driving current to the light device according to a light control signal.

The main controller receives a wireless main control signal from a wall switch operated by a user.

The main controller generates the fan control signal and the light control signal based on the wireless main control signal.

In some embodiments, the main controller includes a wireless receiver.

The wall switch includes a wireless transmitter.

The wireless main control signal is transmitted from the wireless transmitter to the wireless receiver.

In some embodiments, where the wall switch has a manual switch disposed on a wall.

The wall switch has a processor for converting an operation on the manual switch to generate the wireless main control signal.

In some embodiments, the manual switch includes a fan switch and a light switch.

The fan switch and the light switch are separate.

The processor multiplex a fan command from the fan switch and a light command from the light switch to create the wireless main control signal.

In some embodiments, the fan driving current includes three-phase driving currents for driving a three-phase motor of the fan.

In some embodiments, the fan motor is a direct current driven motor.

In some embodiments, the wall switch is also wired to the main controller, when the wireless connection is failed, the wire handles the control signal transmission.

In some embodiments, the wall switch is electrically coupled to the converter to get power to operate.

In some embodiments, there is a heat conductor path between the fan and the light device so that the fan conducts heat dissipation for the light device.

In some embodiments, where there is a heat isolation element between the fan and the light device.

In some embodiments, the lighting apparatus may also include a direction bracket.

The direction bracket is fixed to the integrated housing for adjusting a direction of the light device and the fan.

In some embodiments, a vibration buffer is placed between the fan and the light device to eliminate vibration of the fan to the light device.

In some embodiments, the fan guides air to flow through an air path.

The light device has a ultraviolet light source to disinfect the air in the air path.

In some embodiments, the fan includes an external fan blade and an inner fan blade.

The inner fan blade drives the air to move in the air path.

In some embodiments, the fan has hidden blade inside a fan housing.

The fan has a housing with an air exit for air to move through.

In some embodiments, the fan has a connector for detachably attaching a air guide plate for adjust air flowing direction of the fan.

In some embodiments, the connector is rotatable for adjusting the air flowing direction.

In some embodiments, the main controller has two working modes.

In a first working mode, the fan is rotated in a first speed and in the second working mode, the fan is rotated in a second speed.

The first speed is more than 10 times of the second speed.

The in the second working mode, the light of the light device combined with the fan generates a required dancing pattern.

In some embodiments, the fan has a third working mode to blow air to ground.

The fan has a fourth working mode to suck air from ground.

The main controller controls switching between the third working mode and the fourth working mode.

In some embodiments, the main controller detects an ambient temperature to determine when to automatically turn on the fan.

### BRIEF DESCRIPTION OF DRAWINGS

FIG. 1 illustrates a lighting apparatus with a fan.

FIG. 2 illustrates a system block diagram.

FIG. 3 illustrates another system block diagram.

FIG. 4 illustrates a relation between a wall switch and the lighting apparatus.

FIG. 5 illustrates another lighting apparatus embodiment.

FIG. 6 shows an example of a wall switch.]

FIG. 7 illustrates a type of arrangement between the light device and the fan.

FIG. 8 illustrates another type of arrangement between the light device and the fan.

FIG. 9 shows a bracket that helps changing direction of components.

FIG. 10 shows air path that guides air.

### DETAILED DESCRIPTION

In some embodiments, a lighting apparatus includes a fan 602, a light device 601, an integrated housing 603, a main controller 606, a fan controller 609, a light controller 608 and a converter 605.

The fan controller 609, the light controller 608 and the main controller 606 comprise one or more circuit components to implement the control logic described below. They may be formed as separate modules, partially or completely integrated in one or multiple integrated chips.

The integrated housing 603 is used for disposing the fan 602 and the light device 601.

The converter 605 converts an external AC power 610 to a direct current.

The fan controller 609 is coupled to the converter 605 for generating a fan driving current 611 to the fan 602 according to a fan control signal 612 from the main controller.

Please note that current and signal are invisible and therefore they are not specifically illustrated in every drawing. However, under the description, persons of ordinary skilled in the art should know where the current refer to.

The light controller 608 is coupled to the converter 605 for generating a light driving current 614 to the light device 601 according to a light control signal 613.

The main controller 606 receives a wireless main control signal 615 from a wall switch 607 operated by a user.

The main controller 606 generates the fan control signal 612 and the light control signal 613 based on the wireless main control signal 615.

In some embodiments, the main controller 606 includes a wireless receiver 6061.

The wall switch 607 includes a wireless transmitter 6071.

The wireless main control signal 615 is transmitted from the wireless transmitter 6071 to the wireless receiver 6061.

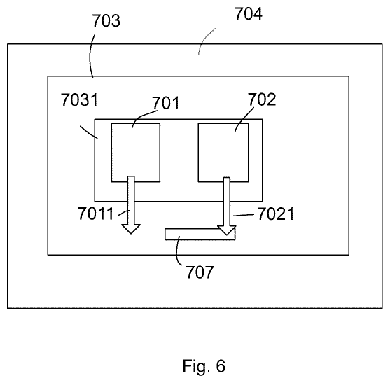

In FIG. 6, where the wall switch 703 has a manual switch 7031 disposed on a wall 704.

The wall switch 703 has a processor 707 for converting an operation on the manual switch 7031 to generate the wireless main control signal.

In some embodiments, the manual switch 7031 includes a fan switch 701 and a light switch 702.

The fan switch 701 and the light switch 702 are separate.

The processor 707 multiplexes a fan command 7011 from the fan switch 701 and a light command 7021 from the light switch 702 to create the wireless main control signal.

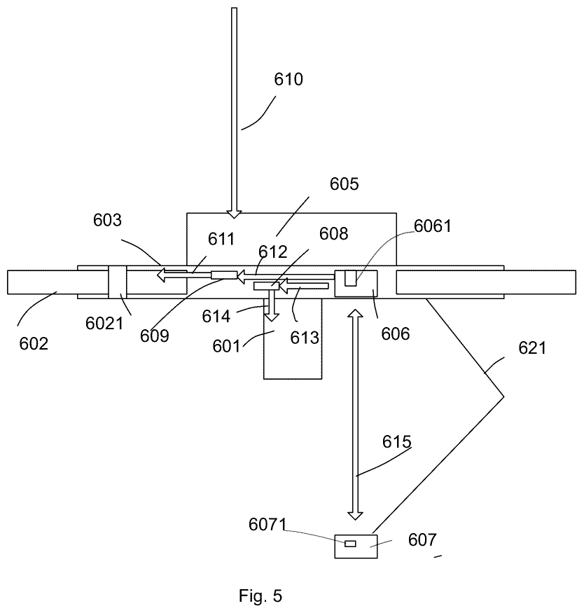

In FIG. 5, the fan driving current includes three-phase driving currents for driving a three-phase motor of the fan.

Three-phase currents are a type of alternating current (AC) used extensively in industrial applications to drive motors, including those used in fans. A three-phase current system consists of three electrical currents that are each out of phase with one another by 120 degrees. This phase difference allows for a continuous transfer of energy, which is especially beneficial for powering motors that require a constant and smooth power supply, such as those used in industrial fans.

When driving a three-phase motor, the three-phase currents are supplied to the motor's windings. These windings are typically arranged in a star (Y) or delta (A) configuration, depending on the motor design and the desired characteristics. As the three-phase currents pass through the windings, they create a rotating magnetic field. This rotating field interacts with the rotor within the motor, inducing a current in the rotor that generates its own magnetic field. The interaction between the stator's rotating field and the rotor's field causes the rotor to spin, thereby converting electrical energy into mechanical energy.

In the case of a fan, this mechanical energy is used to drive the fan blades. As the motor spins the fan blades, air is drawn into the fan and expelled in a specific direction, creating airflow. The direction of the airflow is determined by the design of the fan blades, which are angled to move air efficiently. The speed of the fan can be controlled by adjusting the frequency of the three-phase current supplied to the motor, allowing for precise control of airflow.

Three-phase motors are highly efficient and reliable, making them ideal for applications like fans, where consistent and robust operation is essential. The balanced power distribution in a three-phase system reduces the likelihood of vibrations and mechanical stress, prolonging the motor's lifespan and ensuring smooth operation. Additionally, three-phase systems are more efficient at delivering power over long distances, making them a preferred choice in industrial settings where large fans may be located far from the power source.

Overall, the use of three-phase currents to drive a motor in a fan application is a cornerstone of modern industrial design, providing an effective and efficient solution for moving air in a controlled and reliable manner.

In some embodiments, the fan motor is a direct current driven motor.

In FIG. 5, the wall switch is also wired to the main controller, when the wireless connection is failed, the wire 621 handles the control signals.

In some embodiments, the wall switch is electrically coupled to the converter to get power to operate.

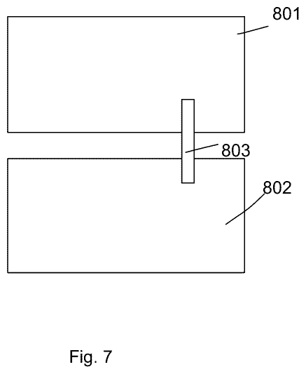

In FIG. 7, there is a heat conductor path 803, e.g. some heat conducting strip, between the fan 801 and the light device 802 so that the fan conducts heat dissipation for the light device 802. Such design uses the fan to conduct heat dissipation to make the light device 802 more stable because it is not overheated.

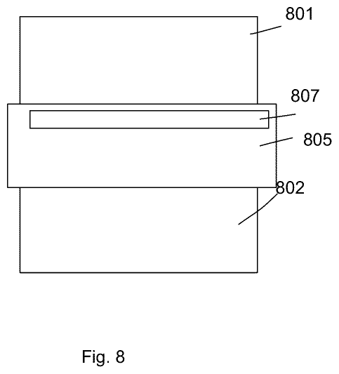

In FIG. 8, where there is a heat isolation element 805 between the fan 801 and the light device 802. In such case, the fan 801 and the light device 802 are thermally separated so that they would not affect each other.

The embodiments in FIG. 7 and FIG. 8 are used under different requirements and may be both useful in certain applications and settings.

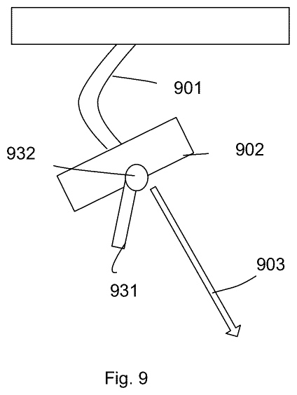

In FIG. 9, the lighting apparatus may also include a direction bracket 901.

The direction bracket 901is fixed to the integrated housing 902 for adjusting a direction 903 of the light device and the fan.

In FIG. 8, a vibration buffer 807 is placed between the fan 801 and the light device 802 to eliminate vibration of the fan to the light device.

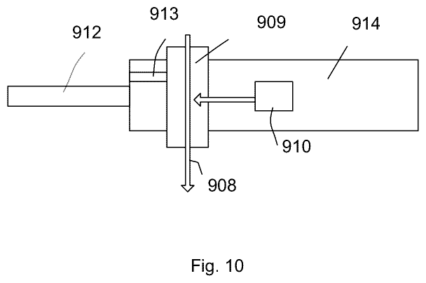

In FIG. 10, the fan guides air 908 to flow through an air path 909.

The light device has an ultraviolet light source 910 to disinfect the air 908 in the air path 909. The air path 909 is hidden in the integrated housing so that the ultraviolet light does not danger to damage user's eyes.

In some embodiments, the fan includes an external fan blade 912 and an inner fan blade 913.

The inner fan blade 913 drives the air to move in the air path 909.

In some embodiments, the fan has hidden blade, e.g. the inner fan blade 913, inside a fan housing 914.

The fan has a housing with an air exit for air to move through.

In FIG. 9, the fan has a connector 932 for detachably attaching an air guide plate 931 for adjust air flowing direction of the fan. It helps when the flow blows air directly on user's head if the fan is installed above the user's seat.

In some embodiments, the connector 932 is rotatable for adjusting the air flowing direction.

In some embodiments, the main controller has two working modes.

In a first working mode, the fan is rotated in a first speed and in the second working mode, the fan is rotated in a second speed.

The first speed is more than 10 times of the second speed.

The in the second working mode, the light of the light device combined with the fan generates a required dancing pattern.

In some embodiments, the fan has a third working mode to blow air to ground.

The fan has a fourth working mode to suck air from ground.

The main controller controls switching between the third working mode and the fourth working mode.

In some embodiments, the main controller detects an ambient temperature to determine when to automatically turn on the fan.

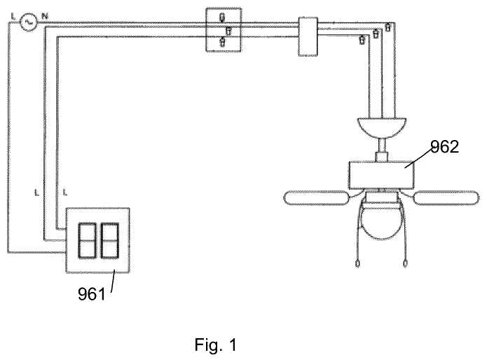

Please see FIG. 1, which shows a lighting apparatus 961 with a fan that is connected to wall switch 961.

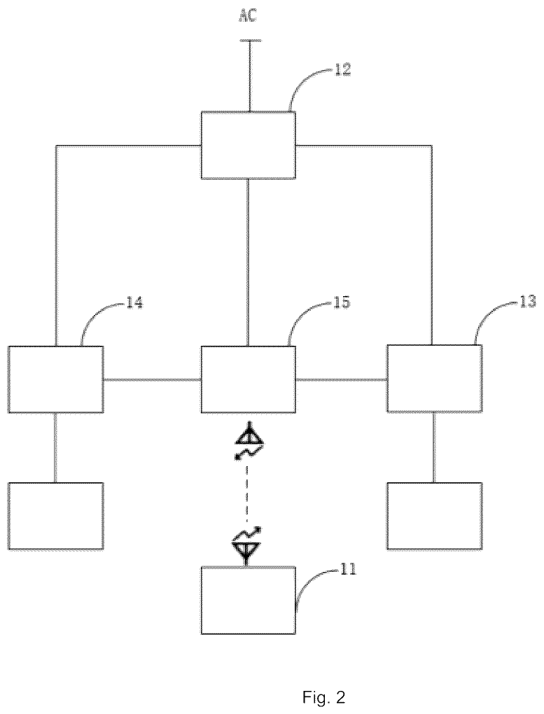

FIG. 2 is a schematic diagram of the structure of a lamp fan control circuit provided by an embodiment of the utility model. Referring to ** FIG. 2 **, the lamp fan control circuit includes: a wall control switch 11, an AC-DC module 12, a lamp driving module 13, a fan driving module 14, and a main control module 15.

The input end of the AC-DC module 12 is used to connect with an AC power supply, and the output end of the AC-DC module 12 is respectively connected to the power input ends of the main control module 15, the lamp driving module 13, and the fan driving module 14, providing power to the main control module 15, the lamp driving module 13, and the fan driving module 14.

The input end of the main control module 15 is wirelessly connected to the wall control switch 11. The first signal output end of the main control module 15 is connected to the control end of the lamp driving module 13, and the second signal output end of the main control module 15 is connected to the control end of the fan driving module 14.

The output end of the fan driving module 14 is used to drive the rotation of the fan, while the output end of the lamp driving module 13 is used to drive the lighting of the lamp.

The wall control switch 11 is used to acquire the first switch action signal from the user and wirelessly transmit this signal to the main control module 15. The main control module 15 receives the first switch action signal and sends a lamp control signal to the lamp driving module 13 or a fan driving signal to the fan driving module 14.

Referring to FIG. 2, in this embodiment, both the lamp driving module 13 and the fan driving module 14 are powered by the AC-DC module 12. The input end of the AC-DC module 12 is connected to the AC power supply (L line and N line). The wall control switch 11 is wirelessly connected to the main control module 15. When the wall control switch 11 is pressed, it generates a first switch action signal. The main control module 15, based on the first switch action signal, controls the lamp driving module 13 or the fan driving module 14 to operate the lamp or fan.

For example, when the lamp switch 111 on the wall control switch 11 is pressed, the main control module 15 receives the first switch action signal, analyzes it, and confirms that it is a lamp-on signal. It then sends a lamp driving signal to the lamp driving module 13 to turn on the lamp.

In this embodiment, a single power line supplies both the lamp and the fan. Only one power line is needed, and the wall control switch 11 does not directly control the power supply to the lamp and fan. Instead, it controls them through the main control module 15, reducing circuit complexity. Additionally, no need exists for a second power line, simplifying wiring and reducing wiring and material costs.

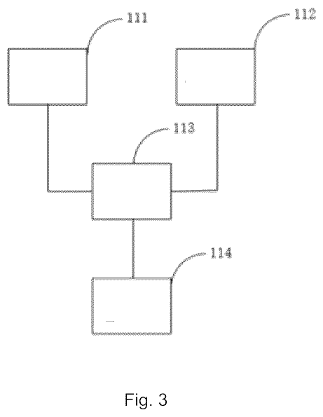

In one possible embodiment, referring to FIG. 3, the wall control switch 11 may include: a lamp switch 111, a fan switch 112, a main control chip 113, and a first wireless communication unit 114.

The first input end of the main control chip 113 is connected to the lamp switch 111, the second input end is connected to the fan switch 112, and the first output end is connected to the first wireless communication unit 114.

In this embodiment, the wall control switch 11 may include both a lamp switch 111 and a fan switch 112. The first switch action signal includes both a lamp switch action signal and a fan switch action signal, which are used to control the lamp and the fan, respectively. Both switches communicate with the main control module 15 through the first wireless communication unit 114, allowing the main control module 15 to receive the lamp switch action signal or the fan switch action signal.

For example, the wall control switch 11 can be mounted on a wall, and the lamp switch 111 and fan switch 112 can be button switches, rocker switches, or touch switches. These switches typically have only two states, used solely to control the on/off status of the lamp or fan.

Additionally, the lamp switch 111 and fan switch 112 can also be rotary switches, which have multiple continuous states. The rotary switch can be used to control the brightness of the lamp and the speed of the fan. The main control chip 113 detects the position information of the rotary switch and sends it to the main control module 15. The main control module 15 then controls the lamp driving module 13 or the fan driving module 14 to adjust the brightness of the lamp or the speed of the fan accordingly.

More specifically, the fan driving module 14 may include a DC-DC unit and a three-phase motor driving unit.

The input end of the DC-DC unit is connected to the power supply end of the fan driving module 14, and the output end of the DC-DC unit is connected to the power supply end of the three-phase motor driving unit.

The control end of the three-phase motor driving unit is connected to the control end of the fan driving module 14, and the three-phase output end of the three-phase motor driving unit is used to output three-phase alternating current (AC) to drive the fan.

The main control module 15 sends a fan control signal to the three-phase motor driving unit based on the action signal from the fan switch 112. The three-phase motor driving unit converts direct current (DC) into three-phase AC (U, V, W) to drive the fan's three-phase motor. For example, if the fan switch 112 is a rocker switch, when the rocker switch is pressed, the fan switch 112 action signal is in the first state, indicating that the fan should start. The main control module 15 then controls the fan to rotate at a preset speed through the fan driving module 14. When the rocker switch is pressed again, the fan switch 112 action signal switches to the second state, indicating that the fan should stop. The main control module 15 then stops the fan through the fan driving module 14.

Alternatively, if the fan switch 112 is a rotary switch, when the rotary switch is turned, the main control chip 113 sends the position information of the fan switch 112 to the main control module 15. The main control module 15 determines the fan speed based on the position information of the fan switch 112 and then controls the output power of the three-phase motor driving unit to adjust the fan speed accordingly. When the rotary switch is returned to its initial position, the fan turns off and stops rotating.

In the above embodiment, the fan driving module 14 is used to drive a three-phase electric fan. When the fan driving module 14 is used to drive a single-phase electric fan, the fan driving module 14 may include a DC-DC unit and a single-phase motor driving unit. The DC-DC unit supplies power to the single-phase motor driving unit, and the control end of the single-phase motor driving unit receives control signals sent by the main control module 15.

The single-phase motor driving unit may be an AC single-phase driving unit, which can control the motor speed using a thyristor for AC speed regulation or control the motor's on/off state through a relay. The specific implementation of these methods is conventional and will not be elaborated here.

The control of the lamp is similar. For example, the lamp driving module 13 can be based on a BUCK architecture, flyback architecture, or a linear constant current architecture.

For instance, the lamp switch 111 can be a rotary switch. When the rotary switch is turned, the main control module 15 controls the output current of the lamp driving module 13 based on the position information of the lamp switch 111, thereby controlling the brightness of the lamp.

The architecture of the lamp driving module 13 is not limited to the ones mentioned above. The BUCK, flyback, and linear constant current architectures are common in the field, and their specific principles will not be further elaborated.

The control logic of the main control module 15 and the main control chip 113 is also not limited to the examples provided above. The control logic is based on existing technology and is not within the scope of protection of this utility model, so it will not be specifically defined here.

It should be noted that the main control module 15 may include a built-in wireless communication module, which communicates with the first wireless communication unit 114. This module may consist of a control chip (e.g., a microcontroller), a wireless communication module, and peripheral circuits.

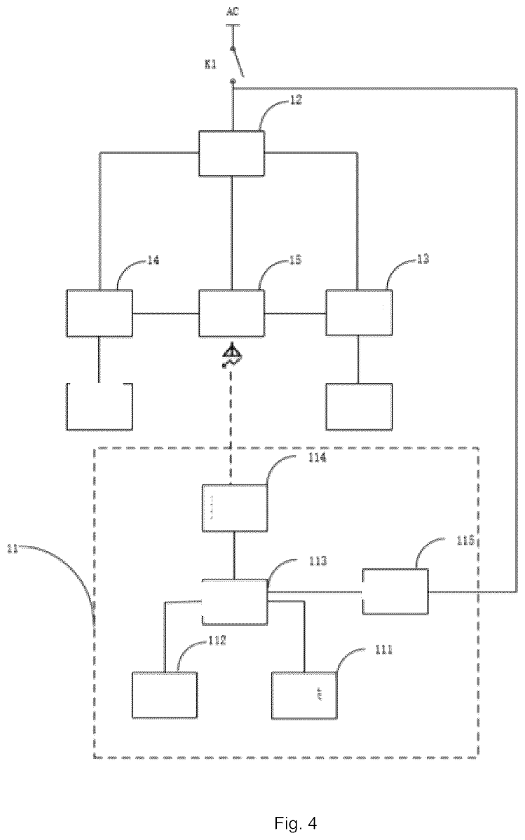

In one possible embodiment, referring to FIG. 4, the wall control switch 11 may also include an AC controllable switch K1.

The AC controllable switch K1 is positioned between the input end of the AC-DC module 12 and the AC power supply.

In this embodiment of the utility model, the AC controllable switch K1 can also be provided. The AC controllable switch K1 can be installed on the same switch panel as the lamp switch 111 and fan switch 112 and connected in series between the AC-DC module 12 and the AC power supply AC to control the power supply from the AC power source.

For example, the AC controllable switch K1 can be a button switch. When the button switch is pressed, the input end of the AC-DC module 12 connects to the AC power supply. When the button switch is pressed again, it resets, and the overall power supply to the lamp and fan is cut off.

In this embodiment of the utility model, the AC controllable switch K1 is provided to control the power supply from the AC source, enhancing the convenience of control and the safety of the lamp and fan.

In one possible embodiment, the wall control switch 11 may also include a battery. The battery is connected to the power supply end of the main control chip 113. This utility model embodiment allows the wall control switch to be powered by a battery.

Since the wall control switch 11 is installed on the wall and the AC power supply runs through the wiring inside the wall, in one possible embodiment, referring to FIG. 4, the wall control switch 11 may also include a power supply unit 115. The input end of the power supply unit 115 is connected to the input end of the AC-DC module 12, and the output end of the power supply unit 115 is connected to the power supply end of the main control chip 113.

The input end of the power supply unit 115 is connected to the input end of the AC-DC module 12, meaning it is connected to the AC power supply. This setup allows the main control chip 113 to be powered by the AC power supply, eliminating the need for a battery, and thus reducing the maintenance effort required for battery replacement.

In another possible embodiment, the lamp fan control circuit may also include a remote controller. The remote controller is wirelessly connected to the input end of the main control module 15. The main control module 15 is further configured to receive a second switch action signal sent by the remote controller.

This embodiment of the utility model may also include a remote controller that wirelessly communicates with the main control module 15. The main control module 15 receives the second switch action signal from the remote controller and controls the operation of the lamp or fan based on this signal. The remote controller and the wall control switch 11 operate in parallel, allowing the state of the lamp switch 111 on the wall control switch 11 to be toggled by the remote controller. If the user wishes to toggle the state again, they can press the lamp switch 111 on the wall control switch 11.

In one possible embodiment, the AC-DC module 12 may include a filtering unit and a rectification unit. The input end of the filtering unit is connected to the input end of the AC-DC module 12, and the output end of the filtering unit is connected to the input end of the rectification unit. The output end of the rectification unit is connected to the output end of the AC-DC module 12.

Corresponding to the above lamp fan control circuit, this utility model embodiment also provides a lamp fan, which includes a lamp, a fan, and the lamp fan control circuit provided in the above embodiments. The lamp fan control circuit is used to drive the lamp to light up and/or the fan to rotate.

The lamp fan includes the lamp fan control circuit provided in the above embodiments and enjoys the advantages of the said lamp fan control circuit, which will not be further detailed here.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings.

The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

1. A lighting apparatus, comprising:
a fan;
a light device;
an integrated housing for disposing the fan and the light device;
a main controller;
a fan controller;
a light controller; and
a converter for converting an external AC power to a direct current, wherein the fan controller is coupled to the converter for generating a fan driving current to the fan according to a fan control signal from the main controller, wherein the light controller is coupled to the converter for generating a light driving current to the light device according to a light control signal, wherein the main controller receives a wireless main control signal from a wall switch operated by a user, wherein the main controller generates the fan control signal and the light control signal based on the wireless main control signal.

2. The lighting apparatus of claim 1, wherein the main controller comprises a wireless receiver, wherein the wall switch comprises a wireless transmitter, wherein the wireless main control signal is transmitted from the wireless transmitter to the wireless receiver.

3. The lighting apparatus of claim 2, where the wall switch has a manual switch disposed on a wall, wherein the wall switch has a processor for converting an operation on the manual switch to generate the wireless main control signal.

4. The lighting apparatus of claim 3, wherein the manual switch comprises a fan switch and a light switch, wherein the fan switch and the light switch are separate, wherein the processor multiplex a fan command from the fan switch and a light command from the light switch to create the wireless main control signal.

5. The lighting apparatus of claim 1, wherein the fan driving current comprises three-phase driving currents for driving a three-phase motor of the fan.

6. The lighting apparatus of claim 1, wherein the fan motor is a direct current driven motor.

7. The lighting apparatus of claim 1, wherein the wall switch is also wired to the main controller, when the wireless connection is failed, the wire handles control signal transmission between the wall switch the main controller.

8. The lighting apparatus of claim 1, wherein the wall switch is electrically coupled to the converter to get power to operate.

9. The lighting apparatus of claim 1, wherein there is a heat conductor path between the fan and the light device so that the fan conducts heat dissipation for the light device.

10. The lighting apparatus of claim 1, where there is a heat isolation element between the fan and the light device.

11. The lighting apparatus of claim 1, further comprising a direction bracket, wherein the direction bracket is fixed to the integrated housing for adjusting a direction of the light device and the fan.

12. The lighting apparatus of claim 1, wherein a vibration buffer is placed between the fan and the light device to eliminate vibration of the fan to the light device.

13. The lighting apparatus of claim 1, wherein the fan guides air to flow through an air path, wherein the light device has a ultraviolet light source to disinfect the air in the air path.

14. The lighting apparatus of claim 13, wherein the fan comprises an external fan blade and an inner fan blade, wherein the inner fan blade drives the air to move in the air path.

15. The lighting apparatus of claim 1, wherein the fan has hidden blade inside a fan housing, wherein the fan has a housing with an air exit for air to move through.

16. The lighting apparatus of claim 1, wherein the fan has a connector for detachably attaching a air guide plate for adjust air flowing direction of the fan.

17. The lighting apparatus of claim 16, wherein the connector is rotatable for adjusting the air flowing direction.

18. The lighting apparatus of claim 1, wherein the main controller has two working modes, wherein in a first working mode, the fan is rotated in a first speed and in the second working mode, the fan is rotated in a second speed, wherein the first speed is more than 10 times of the second speed, wherein the in the second working mode, the light of the light device combined with the fan generates a required dancing pattern.

19. The lighting apparatus of claim 1, wherein the fan has a third working mode to blow air to ground, wherein the fan has a fourth working mode to suck air from ground, wherein the main controller controls switching between the third working mode and the fourth working mode.

20. The lighting apparatus of claim 1, wherein the main controller detects an ambient temperature to determine when to automatically turn on the fan.
